(19) United States
(12) Patent Application Publication
(10) Pub. No.: US 2015/0105704 A1
(43) Pub. Date: Apr. 16, 2015

(54) MEDICAL DEVICE

(71) Applicant: NetScientific Ltd., London, UK (GB)

(72) Inventor: Farad Azima, London (GB)

(21) Appl. No.: 14/403,821

(22) PCT Filed: May 30, 2013
(86) PCT No.: PCT/GB2013/051430

§ 371 Date: Nov. 25, 2014

Related U.S. Application Data
(60) Provisional application No. 61/654,160, filed on Jun. 1, 2012.

(30) Foreign Application Priority Data
Jun. 1, 2012 (GB) 1209762.2

(51) Int. Cl. A61B 18/18; A61N 7/00

(52) U.S. Cl. CPC A61B 18/1815; A61N 7/00
USPC 601/18

(57) ABSTRACT

The invention relates to medical devices and more particularly, but not exclusively, to therapeutic devices for reducing blood pressure.

A device for dilating an artery or other blood vessel or modifying a blood AGE (Advanced Glycation End) is described. The device comprises a dilation stimulation transducer configured for application to the human or animal body and a driver for the dilation stimulation transducer. The driver drives the transducer to output an energetic signal to dilate the artery or other blood vessel.

### FIELD OF THE INVENTION

The invention relates to medical devices and more particularly, but not exclusively, to therapeutic devices for reducing blood pressure.

### BACKGROUND TO THE INVENTION

Hypertension is a major risk factor for strokes, heart attacks, heart failure, aneurisms, and peripheral arterial heart disease and is a cause of chronic kidney disease. Although changes in lifestyle can result in lowered blood pressure, drug treatment is often still necessary.

Our blood pressure is largely controlled by sensors in the carotid artery in the neck, which carries blood to the brain. These signals widen or narrow blood vessels, controlling the pressure.

Researchers at the University of Chicago have shown that blood pressure can be reduced with the aid of electrical stimulation applied to sensors in the carotid artery in the neck. Further studies have also shown that electrical stimulus through baroreflex activation therapy can also be used to lower blood pressure (Baroreflex Activation Therapy Lowers Blood Pressure in Patients with Resistant Hypertension, Journal of the American College of Cardiology, 2011, 58:765-773).

Activating these sensors with electrical stimulation can lower blood pressure in patients who don't respond to medication. Electrodes implanted in the neck send mild electrical pulses to the sensors, which in turn widen the blood vessels and reduce blood pressure.

It is known to use light for a range of different purposes in medicine including cauterisation, wound healing, infra-red tomography, photodynamic therapy and the like. For example it has been found that low level red light at around 630 nm is effective in treating acne. More particularly, US2011/0301671 describes a device for applying laser light to the body for regulating blood pressure. However there are few technical details and the devices described could be difficult to use in practice.

### SUMMARY OF THE INVENTION

It is an object of the invention to provide a non-drug, non-invasive therapeutic device for reducing blood pressure. It is another object of the invention to reduce blood pressure using non-invasive and non-electrical methods.

According to a first aspect of the invention there is therefore provided a device for dilating an artery or other blood vessel or modifying a blood AGE, the device comprising: a dilation stimulation transducer configured for application to the human or animal body; and a driver for said dilation stimulation transducer, to drive said transducer to output an energetic signal to dilate said artery or other blood vessel.

In some embodiments the transducer comprises a light source, which may be a substantially monochromatic light source. Light sources which may be employed include but are not limited to, LEDs (including super luminescent LEDs, and multiple wavelength LEDs) and lasers including, but not limited to, solid state lasers, dye lasers and very short pulse lasers (femtosecond lasers).

In some embodiments the wavelength of the light source may be in the range of 600-2000 nm. The wavelength may be at least 1000 nm, for example 1050 nm or 1340 nm, or 1560 nm (all +1-50 nm), the latter for example, from an Erbium fibre laser. Using light of this wavelength has a number of advantages including reduced scattering through the intervening tissue, and deeper penetration. In other approaches a plurality of different wavelengths may be employed, for example selected taking into account the absorption peaks of chromophores in the body tissue such as one or more of oxyhaemoglobin, deoxyhaemoglobin, water and lipid. Thus, for example, one or more wavelengths may be selected to achieve a desired combination of absorption at the target area and reduced scattering in the intervening tissue. With such an approach one or more wavelengths of the light source may be selected from the range 630-1000 nm. For example one or more of the following wavelengths may be used in the light source (or alternatively may be attenuated or substantially absent form the light source—for example to reduce scattering in tissue by lipid): 650+/−10, 700+/−10, 710+/−10, 716+/10, 720+/−20, 790+/−15, 860+/−20, 890+/−20, 900+/−30, 930+/−20 nm.

In embodiments the laser light may be pulsed, and more particularly may comprise a pulse train, for example at a frequency of greater than 1 KHz, 10 KHz, 100 KHz, 1 MHz, or 10 MHz. Such an approach can be advantageous in achieving penetration/targeting.

In other embodiments the transducer comprises an ultra sonic transducer with a frequency in the range 20 KHz to 200 MHz or greater. Different frequencies within this range may be employed for different purposes: for example the lower frequencies, in particular when focussed, may be employed to deposit energy in a well defined target region—for this purpose frequencies in the range 200 KHz-2000 KHz may be employed. Alternatively where local oscillatory displacement of, for example, the wall of a blood vessel is desired a higher frequency, for example greater than 1 MHz, 2 MHz or 10 MHz may be employed. Optionally more than one frequency may be employed for a combination of effects.

In embodiments two transducers one optical (i.e. a light source) and one acoustic may be employed to provide a combination of acoustic energy and optical energy to the target region. This may have a synergistic effect. (For the avoidance of doubt, such an acoustic source includes, as an example, the ultrasonic transducer and encompasses the broad range of audio signals, not just the audible frequency range).

In still other embodiments the ultra sonic signal may be employed to modulate or otherwise influence the optical output, for example by employing an acousto-optical modulator coupled to an ultrasonic transducer to modulate light from the light source.

In other embodiments the energetic signal may comprise sound, and the driver may comprise an opto-acoustic pulsed laser light source configured to produce an acoustic wave.

In embodiments the active head of the device may comprise a conformal pad, preferably having one or more straps, to allow the transducer(s) to be held against the skin to provide good coupling of energy from a transducer into the skin. The transducer itself may be mounted within the pad or, in an alternative approach, one or more optical fibres may be employed to deliver the light to the pad. For example in embodiments an array of fibres, for example some tens of fibres may be distributed across an output region of the conformal pad. This latter approach makes the pad light and flexible and easy to apply, in particular to a curved portion of the body surface, whilst achieving good skin contact, generally increasing patient convenience.

In some embodiments the transducer (or output region of the pad) has a generally longitudinal configuration, for example an aspect ratio of at least 2:1, 3:1, 5:1, 8:1 or 10:1. This facilitates aligning the stimulation with a major artery. In such a case a visuals indication may be provided on a surface of the pad, for example a dashed line, indicating the orientation of the long axis of the output region. As previously mentioned, in some preferred embodiments the transducer is configured to focus the energetic signal beneath the skin to a point or line focus, or to an approximation to a plane focus, at least one distance beneath the skin in the range 3 mm to 60 mm. Such focusing may employ an optical or acoustic lens or, for example in the case of an ultrasonic transducer, may use a phased array output to provide targeting of the energetic beam.

In some preferred embodiments the device, for example the conformal pad head end, includes an automatic cut-off system to detect removal of the pad from the skin and to reduce or cut-off the energetic signal output in response. This may be provided by a proximity switch (mechanical or electronic, for example capacitative or optical) in the device head, connected to a control/shut off system in the driver. This provides an important safety feature, in particular in the case of an optical output device, especially where the optical output is outside a visible wavelength.

The transducer driver will in general comprise an amplifier, a controller to control the output power, optionally a modulator to modulate the output, and a user interface, preferably coupled to a microprocessor to control the system allow programming of power levels, setting a duration of the treatment to be applied, safety/shut-off features and the like.

The invention also provides a method of treating a human or animal body to dilate an artery or other blood vessel or to modify an AGE (Advanced Glycation End) product in the blood associated with hypertension.

We describe a therapeutic device for dilating an artery or other blood vessel, comprising an exciter in the form of a light source and arranged to irradiate the artery or other blood vessel. The light from the exciter may be pulsating. The light source may comprise an array of lights. The lights in the array may be arranged in a circular pattern. The lights in the array may be arranged in concentric circular patterns.

The wavelength of the light from the light source may be in the range from infrared to ultraviolet. Mid infrared light may be beneficial in applying heat energy to the artery or other blood vessel to cause dilation. The frequency of pulsation of the light source may be in the range from a few cycles per second up to many thousands of cycles per second.

The light source may comprise one or more LEDS or other devices such as low power lasers. The wavelength of the light source may be chosen to improve subcutaneous penetration of the light.

In use, the light exciter of the device may be arranged to be placed in contact with the skin of the patient over an artery to be dilated. The light source may be sized so that accuracy of positioning is not critical. Thus the light source may extend over a diameter of between less than 10 and more than 35 mm and might be in the region of around 25 mm. The artery to be dilated may be the jugular or carotid artery of the neck.

A therapeutic device for dilating an artery or other blood vessel is described, comprising an exciter in the form of a high frequency vibration exciter which may output vibrations in the ultrasound region or in the bordering audible region, and arranged to irradiate the artery or other blood vessel.

A therapeutic device for dilating an artery or other blood vessel is described, comprising an exciter in the form of a light source and arranged to irradiate the artery or other blood vessel in combination with an exciter in the form of a high frequency vibration exciter which may output vibrations in the ultrasound region or in the bordering audible region, and arranged to irradiate the artery or other blood vessel. The ultrasound may be used to modulate and/or otherwise alter the characteristics of light, e.g. to modify the energy and thus to alter characteristics of the wall structure of the artery or other blood vessel to cause the dilation.

A method of dilating a blood vessel, e.g. an artery, is described comprising irradiating the blood vessel with pulsating light and/or with ultrasound. The ultrasound may be such as to modulate the light.

### BRIEF DESCRIPTION OF THE DRAWINGS

These and other aspects of the invention will now be further described, by way of example only, with reference to the accompanying figures in which:

FIG. 1 shows a block diagram of the therapeutic device;

FIG. 2 shows an alternative arrangement of the therapeutic device;

FIG. 3a shows an embodiment combining a laser light source and an acoustic transducer;

FIG. 3b shows an embodiment in which a pulsed laser produces the acoustic signed via an opto-acoustic converter; and

FIG. 4 shows an example of an ultrasonic array that may be used.

### DETAILED DESCRIPTION OF THE PREFERRED EMBODIMENTS

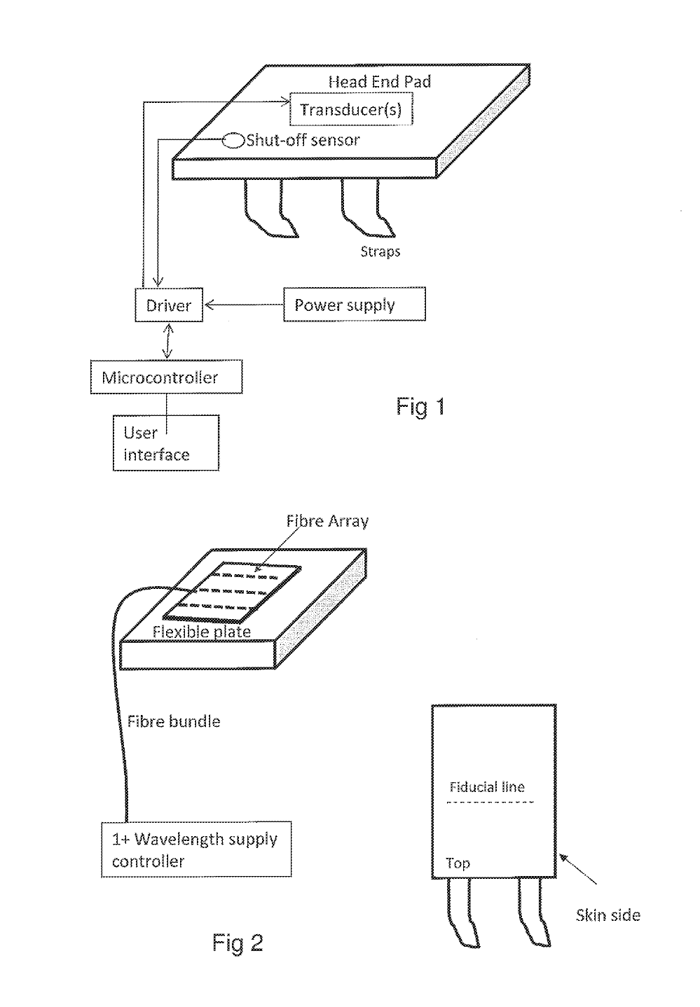

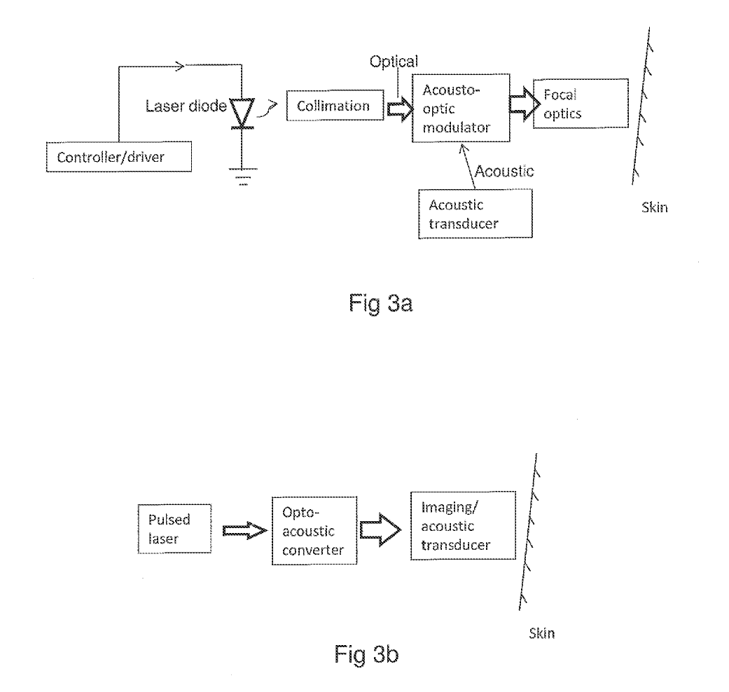

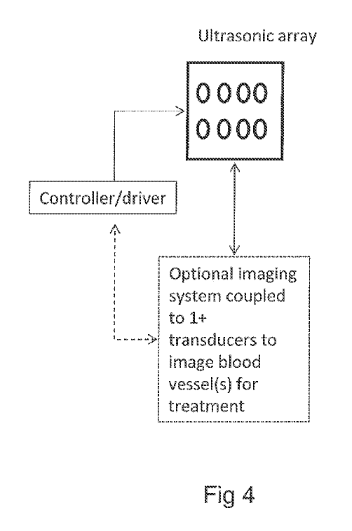

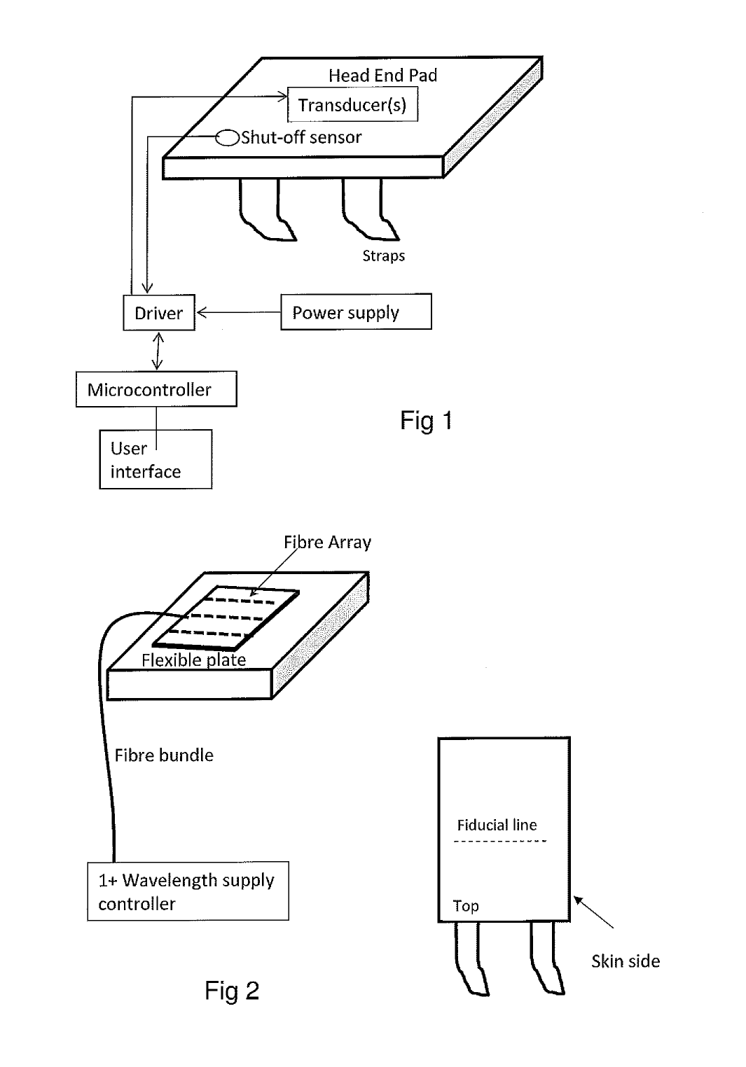

FIG. 1 shows a block diagram of the non-drug, non-invasive therapeutic device for reducing blood pressure. The active head end comprises a conformal pad and one or more dilation stimulation transducers mounted within the pad. The pad is held onto the body using straps to minimise movement of the pad and ensure a good coupling between the transducers and the skin.

The transducer(s) are driven by a transducer driver coupled to a power supply and microcontroller. The driver drives the transducer to output an energetic signal to dilate an artery/blood vessel. User configuration is possible via the user interface, which may be a series of buttons through to a graphical user interface providing instructions and feedback information to the operator.

FIG. 2 shows an alternative arrangement of the therapeutic device. In this example a fibre bundle is used to deliver light to an array of outputs on pad. This allowing the pad to be more flexible, allowing it to be curved or wrapped around a section of person's neck for example and held in place via straps. The output arrangement on the flexible pad allows a wider area to be targeted and allows for automatic adjustment, via the controller, of the targeted area. The fibre array is driven by a controller providing one or more wavelength sources. One examples of a source used to drive the fibre array is an Erbium fibre laser. The laser may be continuous wave and modulated or a short pulsed laser such as a femtosecond laser. The pulse duration may be in the range of 1 to 1000 fs for example.

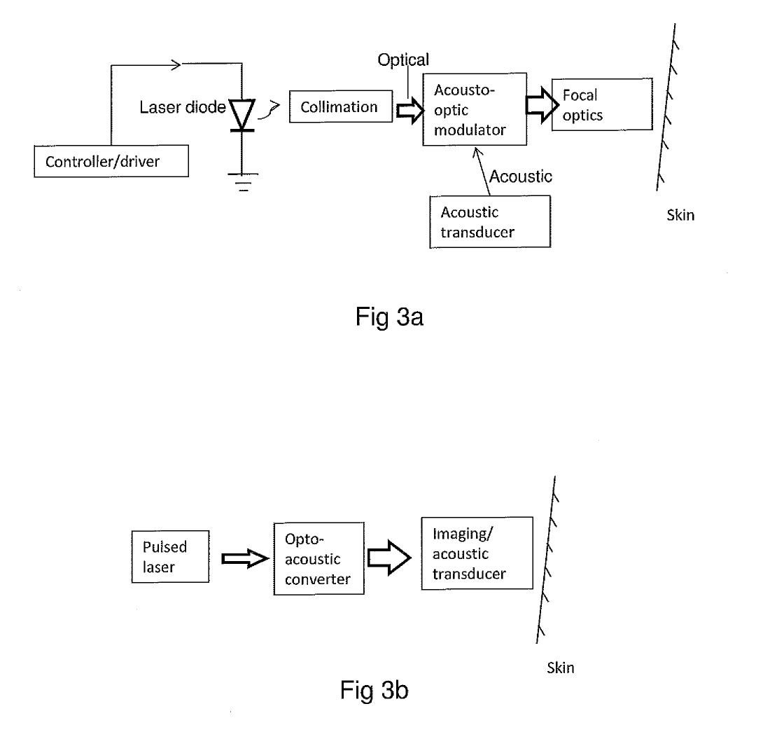

FIG. 3a shows another embodiment of the non-drug, non-invasive therapeutic device for reducing blood pressure. In this embodiment, both a laser light source and an acoustic transducer are used to provide a combination of acoustic energy and optical energy to the target region. In the example shown, a laser diode is used to provide a laser light source which is then subject to collimation. An acousto-optical modulator is then used to control/modulate the light source. The modulated light source is then passed through focussing optics and emitted onto the skin. The focussing optics can be used to target the focal point of the energetic beam to different depths below the skin.

FIG. 3b shows a further embodiment of the non-drug, non-invasive therapeutic device for reducing blood pressure. In this embodiment, a pulsed laser is used to generate a laser light source, which is then used to produce an acoustic signal via an opto-acoustic converter. In this variant, no separate acoustic transducer may be needed.

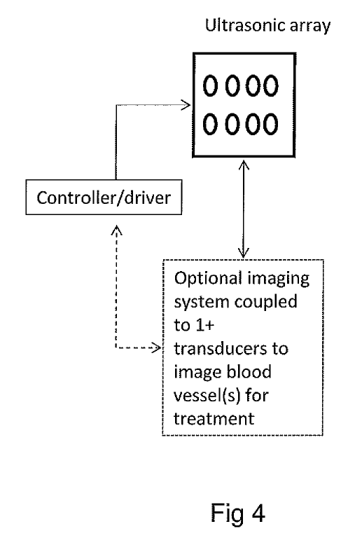

Referring now to FIG. 4, this show another variant which further uses an ultrasonic array driven by a controller/driver circuit or an optical coherent tomography (OCT) imaging device. The ultrasonic array/OCT may be used provide imaging of an area of the body. When combined with features of the previous embodiments, such as the transducer(s) and/or fibre array, the pad attached to a person's body can be used to both image blood vessels and provide treatment.

This arrangement may also be particularly useful when combined with one of the previous examples in order to actively monitor the area being treated and monitor and detect for movement/slippage and any misalignment of the pad and generated energetic signal. Following detection, one option may then be to feedback this information to the operator, via the user interface, or alternatively automatic adjustment may be possible be selecting different outputs from the fibre array or refocusing the emitted signal.

The invention described herein has been demonstrated for its application to reducing blood pressure. It will be appreciated that the device and any such methods of treatment may also be applicable to other non-invasive therapeutic treatments responsive to such stimulation technology described herein, in particular those that have been shown to be responsive to electrical stimulation. These include the control of weight, whereby application to the area of the brain that controls fullness may help to assist people eat less; improving the health of heart failure patients by stimulating blood flow (patients may be physically inactive); cystisis; tinnitus; diabetes; asthma; dementia; and depression.

No doubt many other effective alternatives will occur to the skilled person. It will be understood that the invention is not limited to the described embodiments and encompasses modifications apparent to those skilled in the art lying within the spirit and scope of the claims appended hereto.

Through out the description and claims of this specification, the words “comprise” and “contain” and variations of the words, for example “comprising” and “comprise”, means “including but not limited to, and is not intended to (and does not) exclude other moieties, additives, components, integers or steps.

1. A device for dilating an artery or other blood vessel or modifying a blood AGE, the device comprising:
a dilation stimulation transducer configured for application to the human or animal body; and
a driver for said dilation stimulation transducer, to drive said transducer to output an energetic signal to dilate said artery or other blood vessel.

2. The device of claim 1, wherein said energetic signal comprises light and wherein said transducer comprise a light source, in particular a substantially monochromatic light source.

3. The device of claim 2, wherein said light source has a wavelength in the range of 600-2000 nm.

4. The device of claim 2 wherein said light source has a wavelength of at least 1000 nm.

5. The device of claim 2 wherein said light source has a plurality of wavelengths comprising at least two wavelengths of absorption chromophores selected from the set comprising oxyhaemoglobin, deoxyhaemoglobin, water and liquid.

6. The device of claim 2 wherein said driver is configured to pulse said light source.

7. The device of claim 1 wherein said transducer comprises an ultrasonic transducer.

8. The device of claim 7 wherein said energetic signal comprises sound and wherein said driver and transducer are configure to provide sound having a frequency comprising one or more of:
a frequency in the range of 1 to 20 MHz; and
a frequency in the range of 200 kHz to 2 MHz.

9. The device of claim 1, wherein said dilation stimulation transducer comprises first and second said transducers comprising, respectively, a light source and an acoustic source.

10. The device of claim 9 further comprising an acousto-optical modulator to modulate light from said light source using said acoustic source.

11. The device of claim 1, wherein said energetic signal comprises sound, and wherein said driver comprises an opto-acoustic pulsed laser light source configured to produce an acoustic wave.

12. The device of claim 1 further comprising a conformal pad, preferably having one or more straps, wherein said transducer is within said pad and wherein said pad is configured to hold said transducer against the skin of said body.

13. The device of claim 12 further comprising an automatic cut-off system to detect removal of said pad from said skin and to reduce output of said energetic signal in response.

14. The device of claim 1 wherein said transducer has an output region having an aspect ratio of at least 3:1, 5:1 or 10:1.

15. The device of claim 14 when dependent on claim 10 wherein said transducer is on a lower surface of said pad and wherein an upper surface of said pad is provided with an indication of a direction of a long axis of said output region of said transducer.

16. The device of claim 1 wherein said transducer is configured to focus said energetic signal beneath the skin to at least one focal distance in the range of 3 mm to 60 mm.

17. A method of treating a human or animal body to dilate an artery or other blood vessel or modify an AGE, the method comprising the steps of:
applying a dilation stimulation transducer to a human or animal body; and
driving said dilation stimulation transducer to output an energetic signal to stimulate said artery or other blood vessel or modify an Advanced Glycation End-products (AGE), to cause said artery or other blood vessel or modify an AGE to dilate.

18. (canceled)

19. The method of claim 17 wherein said energetic signal comprises light and wherein said transducer comprise a light source, in particular a substantially monochromatic light source.

20. The method of claim 19 further comprising the step of pulsing said light source.

21. The method of claim 20 wherein said energetic signal comprises sound having a frequency comprising one or more of:
a frequency in the range of 1 to 20 MHz; and
a frequency in the range of 200 kHz to 2 MHz.
